(19) United States
(12) Patent Application Publication
(10) Pub. No.: US 2023/0338475 A1
(43) Pub. Date: Oct. 26, 2023

(54) USE OF INHALED INTERFERON-BETA TO IMPROVE OUTCOME IN SARS-CoV-2 INFECTED PATIENTS

(71) Applicant: SYNAIRGEN RESEARCH LIMITED, Southampton Hampshire (GB)

(72) Inventors: Richard MARSDEN, Southampton Hampshire (GB); Phillip MONK, Southampton Hampshire (GB); Tom WILKINSON, Southampton Hampshire (GB)

(73) Assignee: SYNAIRGEN RESEARCH LIMITED, Southampton Hampshire (GB)

(21) Appl. No.: 18/016,964

(22) PCT Filed: Jul. 19, 2021
(86) PCT No.: PCT/GB2021/051853

§ 371 Date: Jan. 19, 2023

(30) Foreign Application Priority Data
Jul. 20, 2020 (GB) 2011216.5
Jul. 21, 2020 (GB) 2011284.3
Apr. 27, 2021 (GB) 2106014.0

(51) Int. Cl. A61K 38/21; A61K 9/00; A61K 45/06; A61P 31/14

(52) U.S. Cl. CPC A61K 38/215; A61K 9/0078; A61K 45/06; A61P 31/14

(57) ABSTRACT

The present invention relates to use of inhaled interferon-beta for use in preventing or reducing the severity of lower respiratory tract (LRT) illness in a patient infected with a coronavirus capable of causing acute respiratory distress syndrome, e.g. preventing or reducing the severity of LRT illness in COVID-19 patients infected with SARS-CoV-2. Targeting of such treatment to promote recovery based on simple and quick breathlessness scoring is proposed which is applicable in both the home and hospital environments.

### FIELD OF THE INVENTION

The present invention relates to use of inhaled interferon-beta (IFN-β), e.g. formulated for nebuliser administration via the airways, to improve outcome in SARS-CoV-2 virus infected patients by preventing or reducing the severity of lower respiratory tract (LRT) illness and/or improving symptom status according to commonly employed scales utilised in clinical practice worldwide for assessing illness severity arising from such infection (commonly referred to as COVID-19). On the basis of trials extending to the home environment, severity of breathlessness is proposed as a simple point of care criterion to be used as an indicator for inhaled IFN-β administration to SARS-CoV-2 infected patients. Such administration to SARS-CoV-2 infected patients with marked or severe breathlessness has been found to significantly accelerate recovery and prevent deterioration and hence may assist in reducing the need for hospitalisation. Although the foundation of the invention has been a clinical trial of inhaled IFN-β with SARS-CoV-2 infected patients, it will be appreciated that the invention has application to any future emerging coronavirus which has the capability to cause severe acute respiratory syndrome and hence categorised as a SARS virus and other known or future emerging coronaviruses, or other potentially pandemic-causing viruses, which cause serious LRT illness in humans.

### BACKGROUND TO THE INVENTION

IFN-β driven anti-viral responses have been shown to be compromised/deficient in older people and those with chronic airway diseases, more particularly asthma and COPD (Agrawal et al. (2013) Gerontology 59, 421-426; Wark et al. (2005) J. Exp. Med. 937-47; Singanavagam et al. (2019) Am. J. Physiol. Lung Cell Mol. Physiol. 317 (6): L893-L903). This is in keeping with previous proposed use of inhaled IFN-β to treat virus-induced exacerbation of asthma and chronic obstructive pulmonary disease (COPD) by common cold-causing rhinoviruses (see EP1734987B in the name of University of Southampton and exclusively licensed to Synairgen plc) and previous proposed use of inhaled IFN-β to reduce severity of LRT illness in the elderly arising from rhinovirus infection (See U.S. Pat. No. 7,871,603B in the name of Synairgen Research Limited). Additionally, EP2544705B, also in the name of Synairgen Research Limited, proposes use of inhaled IFN-β for treatment of LRT illness associated with influenza infection. Clinical trials using an inhaled IFN-β1a formulation for nebulisation delivered via a breath actuated nebuliser have been conducted to further such administration especially in asthmatics or COPD patients suffering LRT illness through a cold or influenza with encouraging results. In all such clinical trials (3 in asthma and one in COPD patients) conducted to date, inhaled IFN-β has upregulated lung antiviral biomarkers in sputum for 24 hours after dosing, confirming successful delivery of biologically active drug to the lungs, demonstrating proof-of-mechanism, and supporting dose selection. However, such trials provide no foundation for assuming that inhaled IFN-β can have any beneficial effect in preventing or treating severe LRT illness associated with any coronavirus capable of causing severe acute respiratory syndrome such as SARS-CoV-2.

IFN-β has been shown to inhibit replication of various coronaviruses including Middle East Respiratory Syndrome-coronavirus (MERS-CoV), SARS-CoV and SARS-CoV-2 in cell-based assays. For example, in vitro studies using Vero cells, which cannot express Type 1 interferons in response to infection, demonstrated that either IFN-β or IFN-α have anti-viral activity against SARS-CoV-2, similar to findings for SARS-CoV and MERS virus (Mantlo et al. Antiviral activities of type I interferons to SARS-CoV-2 infection. Antiviral Res. 2020;179:104811). CN1535724A (Beijing Jindike Biotech. Res. Institute) also reports cellular assays with various Type I interferons indicating ability to inhibit SARS virus proliferation, but if anything favours focus on IFN-α2b. Extension of those studies is reported in CN1927389A which reports use of a recombinant human IFN-α2b nasal spray as a preventive measure against SARS virus infection in the upper respiratory tract. However, no Type I interferon formulation suitable for inhaled delivery is taught; the only formuation of recombinant human IFN-β mentioned is a commercial formulation to be injected. In the light of such studies, it has remained unknown whether inhaled IFN-β can have any benefit in preventing or treating LRT illness in patients infected with any coronavirus, especially providing improved symptom status/outcome in such patients with LRT illness warranting categorisation of at least 3 or 4 on the WHO recognised Ordinal Scale for Clinical Improvement in relation to illness arising from SARS virus infection and need for hospitalisation.

Indeed, the only reported clinical trial employing IFN-β in patients with confirmed COVID-19 disease arising from SARS-CoV-2 infection only looked at subcutaneous injection of IFN-β-1b in a formulation not even suitable for inhalation administration and in combination with oral administration of lopinavir-ritonavir and ribavirin (see Hung et al. (2020) Lancet 395: 1695-16704 entitled ‘Triple combination of interferon beta-1b, lopinavir-ritonavir and ribavirin in the treatment of patients admitted to hospital with COVID-19: an open-label, randomised, phase 2 trial’; first published on-line 8 May 2020). The same clinical trial team only suggest that double anti-viral therapy with IFN-β-1b is warranted as employed for the triple combination therapy studied. Patients in the reported trial were randomly assigned to either the triple combination therapy group or a group given only lopinavir-ritonavir. In the triple combination group, patients were recruited and treated at less than 7 days from symptom onset with IFN-β-1b; they received one to three doses of IFN-β-1b on alternate days depending on the day of drug commencement. If commenced on day 1-2 from symptom onset, the patient received all 3 doses of IFN-β. If drug administration was commenced on day 3-4 from symptom onset, the patient received two doses of IFN-β. If drug administration was commenced on day 5-6 from symptom onset, the patient received one dose of IFN-β. For those treated between days 7 and 14, IFN-β was not administered at all. Hence, IFN-β is suggested at best for early intervention in COVID-19 disease by subcutaneous injection with indicated concern over whether such injection may have undesirable effect in relation to symptom development linked to inflammatory mechanisms beyond the initial viral infection.

Preceding studies using IFN-β to treat MERS-infected mice demonstrated that the treatment was only effective if given within one day of infection, before the virus load peaked, whereas delayed interferon treatment failed to inhibit virus replication and caused increased inflammation and enhanced proinflammatory cytokine expression leading to the suggestion that delayed treatment could exacerbate symptoms by causing a cytokine storm (see Channappanavar et al. IFN-I response timing relative to virus replication determines MERS coronavirus infection outcomes. J. Clin. Invest. 2019;129 (9):3625-39). This supports if anything early treatment with IFN-β for COVID-19 (and by injection) consistent with the above-noted human clinical trial.

The rationale for confining use of IFN-β to early intervention in this clinical situation (prior to 7 days from on-set of symptoms) can be seen as expectation that SARS-CoV-2 viral load will peak within a few days of symptom onset coupled with concern that later treatment might drive the cytokine storm seen in COVID-19 patients and observed in the animal model of MERS virus infection. Indeed, it has been proposed that the cytokine storm and the damage it causes may be a more important driver of severe disease than viral replication during the later stages of the disease and that secondary induction of interferons may drive the cytokine storm (Andreakos E. & Tsiodras S. COVID-19: Iambda interferon against viral load and hyperinflammation. EMBO Mol. Med. 2020;12 (6):e12465; Jamilloux et al. Should we stimulate or suppress immune responses in COVID-19? Cytokine and anti-cytokine interventions. Autoimmun. Rev. 2020;19 (7):102567).

A team at the Amiens-Picarde University Hospital, France, in a review article entitled ‘Therapeutic Options for Coronavirus Disease 2019 (COVID-19)—Modulation of Type I Interferon Response as a promising Strategy?’ (Mary et al (May 2020) Frontiers in Public Health, 8, Article 185) have recently commented on possible approaches to treatment of COVID-19 disease. They note the well-publicised clinical use in China of nebulization administration of IFN-alpha-2b (see further information on IFN-alpha below) with a view to treating LRT illness associated with SARS-CoV, Middle East respiratory Syndrome Coronavirus (MERS virus) and more recently SARS-CoV-2, but provide no new data supporting similar delivery of IFN-β with beneficial effect at any stage of COVID-19 disease. They merely note preliminary information on the above-noted triple combination drug trial and if anything point to azithromycin representing an interesting alternative strategy for investigation. Apart from its anti-bacterial role, azithromycin has been reported to increase rhinovirus-induced Type I and Type II IFN response in bronchial epithelial cells from healthy donors, asthmatic individuals and patients with COPD. Combination therapy of azithromycin and hydroxychloroquine has been trialled in some French COVID-19 patients (Gautret et al. Int. J. Antimicrob. Agents (2020) 105949) and Mary et al. comment that “the possibility must be considered that azithromycin may be responsible for the rapid reduction of viral carriage in this sub-group of h-CQ-treated French patients.”

An article by Sallard et al. entitled ‘Type I interferons as potential treatment against COVID-19’ (Antiviral Research, 178, 104791, on-line 7 Apr. 2020) reviews various studies relevant to judging benefit of use of Type I interferons, both IFN-α and IFN-β, in relation to viral-induced ARDS. As regards inhalation administration, the discussion is again confined to administration of IFN-α and the common recommendation for such administration of IFN-α as part of combination therapy, e.g. in combination with lopinavir/ritonavir (See, for example, Lu, H. Drug treatment options for the 2019-new coronavirus (2019-nCoV) Bioscience Trends (2020) 14 (1); 69-71; Dong et al. Discovering Drugs to treat coronavirus disease 2019 (COVID-19), Drug Discover. Therap. (2020) 14 (1): 58-60; Liu et al. Critical care response to a hospital outbreak of the 2019-nCoV infection in Shenzhen, China; Crit. Care (2020) 24-56). That such reports on use of inhaled IFN-α do not speculate about any use of IFN-β is notable and consistent with knowledge on Type I interferons.

Although Type I interferons such as IFN-α and IFN-β are anti-viral proteins that bind to the same receptor, they differ in their anti-viral and immunomodulatory properties (Ng et al. Alpha and Beta Type 1 Interferon Signaling: Passage for Diverse Biologic Outcomes. Cell, 2016;164 (3):349-52; Gibbert et al. IFN-alpha subtypes: distinct biological activities in anti-viral therapy. Br. J. Pharmacol. 2013;168 (5):1048-58). Cells produce interferons as an innate immune response to combat a viral infection. It is this innate immune response that provides a first line of defence against viruses until the adaptive immune system generates antibodies and cell mediated responses, which clear the virus infection and can provide long term immunity. IFN-α is produced in large quantities by specialised white blood cells called plasmacytoid dendritic cells and it is approved for use in some systemic infections such as hepatitis. IFN-β is made by many cell types, including epithelial cells and fibroblasts where it is produced as an immediate local response to viral infection and triggers an antiviral programme preparing the tissue to fight off the infection. It has been reported that IFN-β is a more potent inhibitor of coronaviruses than IFN-α in cellular studies (see again the above-noted reference of Mantlo et al.; see also Scagnolari et al. Increased sensitivity of SARS-coronavirus to a combination of human type I and type II interferons. Antiviral Ther. 2004;9 (6):1003-11 and Stockman et al. SARS: systematic review of treatment effects. PLoS Med. 2006;3 (9):e343). However, it will be appreciated that such cellular studies do not enable prediction of benefit of inhaled IFN-β in the complex clinical picture of coronavirus infection-induced acute lower respiratory illness.

The only reference in the above-noted Sallard et al. review to IFN-β administration is to administration by injection and again there is expressed need for any Type I interferon to be administered as early as possible to optimise antiviral therapy and avoid adverse events with reference to the above-noted 2019 report of Channappanavar et al. Indeed, the authors go as far as to suggest that in the late phases of COVID-19 progression associated with severe LRT illness, it may be more beneficial to administer anti-interferon drugs.

Viral infection naturally triggers an innate immune response involving production of Type I and II interferons which induce antiviral genes and regulate inflammatory responses. However, like the highly homologous SARS-CoV, SARS-CoV-2 possesses an array of non-structural proteins that can prevent both host expression of Type I interferons, as well as downstream signalling from the Type I interferon receptor (Kindler et al. Interaction of SARS and MERS Coronaviruses with the Antiviral Interferon Response. Adv. Virus Res. 2016;96:219-43; Yuen et al. SARS-CoV-2 nsp13, nsp14, nsp15 and orf6 function as potent interferon antagonists. Emerging Microbes Infect. 2020;9 (1):1418-28). Consistent with this, severe COVID-19 patients have been reported to show impaired Type I interferon activity and inflammatory responses (Hadjadj et al. Impaired type I interferon activity and inflammatory responses in severe COVID-19 patients. Science (2020) 369, 718-724).

While as discussed above there has been considerable emphasis on early administration of interferons to reduce the peak of viral replication, it has been reported that patients with high viral loads and long virus-shedding periods are at higher risk of severe COVID-19 with severe LRT illness (Liu et al. Viral dynamics in mild and severe cases of COVID-19. Lancet Infect. Dis. 2020; 20 (6): 656-7). Furthermore, the use of inhaled IFN-β for intervention at any time point has not been suggested.

### SUMMARY OF THE INVENTION

Now presented for the first time is data supporting use of inhaled IFN-β for preventing or reducing the severity of LRT illness associated with SARS-CoV-2 infection and/or improving symptom status/treatment outcome for patients even when presenting with at least a score of 3 or at least 4 for COVID-19 illness associated with SAR2-CoV-2 infection on the WHO recognised Ordinal Scale for Clinical Improvement as set out immediately below (see also WHO R&D Blueprint novel Coronavirus COVID-19 Therapeutic Trial Synopsis. Feb. 18, 2020).

### Ordinal Scale for Clinical Improvement

| Patient State | Descriptor | Score |
|---|---|---|
| Uninfected | No clinical or virological | 0 |
| | evidence of infection | |
| Ambulatory | No limitation of activities | 1 |
| | Limitation of activities | 2 |
| Hospitalised | Hospitalised, no oxygen | 3 |
| Mild disease | therapy | |
| | Oxygen by mask or nasal | 4 |
| | prongs | |
| Hospitalised | Non-invasive ventilation or | 5 |
| Severe disease | high-flow oxygen | |
| | Intubation and mechanical | 6 |
| | ventilation | |
| | Ventilation + additional | 7 |
| | organ support - pressors, | |
| | RRT, ECMO | |
| Dead | Death | 8 |

Where the Ordinal Scale of Clinical Improvement (OSCI) is referred to hereinafter it will be understood to refer to the scoring system as given above.

Data is additionally now reported from administration of inhaled IFN-β to SARS-CoV-2 infected patients in the home setting which has shown that such IFN-β administration can speed recovery, defined as level 1 or 0 on the above-noted Ordinal Scale, in such patients who at the start of IFN-β treatment are exhibiting marked or severe breathlessness. Breathlessness was scored using a recognised scoring system based on patient response to the question: how much difficulty did you have breathing today?
0=None—unaware of any difficulty
1=Mild—noticeable when performing strenuous activity (e.g. running)
2=Moderate—noticeable even when performing light activity (e.g. bedmaking or carrying groceries)
3=Marked—noticeable when washing or dressing
4=Severe—almost constant, present even when resting

Such breathlessness scoring forms an element of the BCSS scoring system previously devised for assessing severity of respiratory disease of COPD patients (Leidy et al. (2003) Chest, 124, 2182-2191: ‘The Breathlessness, Cough and Sputum Scale. The Development of Empirically Based Guidelines for Interpretation’). Where breathlessness score is hereinafter referred to it will be understood to refer to scoring of breathlessness in this recognised manner.

The ability to speed recovery was most marked in SARS-CoV-2 infected individuals showing breathlessness of greater than or equal to 3 on this scale, i.e. exhibiting marked or severe breathlessness. In both the hospital and home cohorts, patients with marked or severe breathlessness were markedly slower to recover than patients with scores of 0-2 on the breathlessness scale. Inhaled IFN-β treatment accelerated recovery in patients with marked or severe breathlessness in both the home and hospital cohort studies.

Thus, the present invention provides IFN-β for use in preventing or reducing the severity of lower respiratory tract illness in a patient infected with a coronavirus capable of causing acute respiratory distress syndrome (ARDS), e.g. severe acute respiratory syndrome commensurate with categorisation as a SARS virus, and/or improving one or more symptoms and/or outcome in a patient so infected, wherein the IFN-β is administered by inhalation.

As indicated above, in the case of SARS-CoV-2 infected patients, breathlessness is a symptom which can both contribute to severity of illness leading to hospitalisation but can also be a noticeable problem in SARS-CoV-2 infected individuals even in the home setting, i.e. for example at score 2 on the WHO Ordinal Scale (noticeable limitation effect on normal activities). Marked or severe breathlessness, i.e. having a breathlessness score of 3-4 on the above-noted scale, is now proposed as a preferred criterion for targeting patients with viral infection of the type of note herein with a view to not only preventing deterioration, but promoting recovery. Thus, a means for targeting effective inhaled IFN-β treatment in relation to COVID-19 disease (or similar viral disease) is proposed based on simple point of care breathlessness scoring which renders such treatment an option even in the home environment with a view to accelerating recovery and preventing need for hospitalization.

The invention will be hereinafter principally described with reference to SARS-CoV-2 and the relevant clinical trial information relating to COVID-19 patients herein presented but the invention is seen by reasonable extrapolation as having application to any known or future emerging coronavirus (or other possibly pandemic-causing virus) which has the capability to cause acute respiratory distress syndrome and other known or future emerging coronaviruses which cause serious LRT illness in humans, e.g. other known SARS viruses, the MERS virus and possible future emerging coronaviruses or other pandemic viruses capable of causing LRT illness in humans arising for example by zoonotic transfer.

Thus in its broadest aspect, the present invention may be seen as providing IFN-β for use in preventing or reducing the severity of lower respiratory tract illness in a patient infected with a virus capable of causing acute respiratory distress syndrome (ARDS) and/or improving one or more symptoms and/or outcome in a patient so infected, wherein the IFN-β is administered by inhalation. The virus may be a coronavirus, e.g. SARS-CoV-2 which causes COVID-19 disease in humans (to be regarded as including any known or future emerging SARS-Cov-2 variant e.g. any variant designated by Greek alphabet designation in accordance with the WHO notice of 31 May 2021). It may be a future emerging virus, for example, arising by zoonotic transfer and potentially capable of causing similar ARDS in humans.

By a virus capable of causing serious LRT illness, which might for example be alternatively termed acute respiratory distress syndrome, will be understood a virus having the capability to cause LRT illness indicating need for hospitalisation and possibility of progression to need for oxygen therapy by mask or nasal prongs according to the WHO Ordinal Scale for Clinical Improvement at least in a sub-group of otherwise healthy people or people having an underlying non-virus related health condition. In the case of coronaviruses categorised as SARS, it will be appreciated that such LRT illness may be commonly termed severe acute respiratory syndrome equating with possible still higher scores according to the same scale.

An aim of administration of inhaled IFN-β according to the invention can be to prevent a patient with a SARS-infection, or SARS-type viral infection capable of causing commensurate LRT illness, progressing from a score associated with mild disease to a score associated with severe disease or progressing from one level of severe disease up to a still higher level of severity, e.g. up to requiring mechanical ventilation. The data now provided illustrates how such administration may be associated with improved outcome in terms of preventing increased progression of severity of LRT disease compared with patients receiving a placebo control and higher likelihood of such patients leaving hospital and perhaps sooner. Indeed, as noted above, administration of inhaled IFN-β treatment on the basis of breathlessness score is now presented as a simple means of targeting such treatment in a manner which has shown significantly accelerated recovery (down to 0 or 1 on the OSCI scale) in both hospitalised and non-hospitalised patients with SARS-CoV-2 infection.

Effectiveness of the inhaled IFN-β may additionally or alternatively be monitored in terms of improvement of one or more symptoms, for example breathlessness. For example, improvement may be assessed in known manner by the Breathlessness, Cough and Sputum scoring (BCSS) system noted above, or the breathlessness scoring element of this. Further details are provided in the exemplification below.

The invention is described further below by reference to the clinical trial data provided in the exemplification and illustrated by the figures as described below.

### DESCRIPTION OF THE FIGURES

FIG. 1:

Recovery of the hospital cohort of patients. Patients who received SNG001 (n=48) were more than twice as likely to recover form COVID-19 (defined as ‘no limitation on activities’ or ‘no clinical or virological evidence of infection’, i.e. level 1 or 0 on the OSCI scale) as those receiving placebo (n=49) (HR 2.19 [95% CI 1.03-4.69]; p=0.043).

FIG. 2:

Recovery of patients with more severe disease at time of admission (requiring treatment with supplemental oxygen). Patients treated with SNG001 (n=36) were more than twice as likely to have recovered by the end of the treatment period (HR 2.60 [95% CI 0.95-7.07]; p=0.062), and had greater odds of recovery at day 28 (OR 3.86 [95% CI 1.27-11.75]; p=0.017) than the placebo group (n=28). Recovery is defined as ‘no limitation on activities’ or ‘no clinical or virological evidence of infection’.

FIG. 3:

Discharge from hospital in patients with more severe disease at time of admission (requiring treatment with supplemental oxygen). SNG001 (n=36) treatment increased the likelihood of hospital discharge (HR 1.72 [95% CI 0.91-3.25]; p=0.096) compared to placebo (n=28). Median time to discharge was 6 days for patients treated with SNG001 and 9 days for patients who received placebo.

FIG. 4:

Breathlessness change from baseline of hospital cohort of patients. Over the treatment period, breathlessness (assessed on the 5-point breathlessness score scale by the patient) was markedly reduced in patients who received SNG001 (n=46) compared to patients who received placebo (n=49) (Difference −0.6 [95% CI −1.0 to −0.2]; p=0.007).

FIG. 5:

Recovery for the home cohort of SARS-CoV-2 infected patients with breathlessness (11%) at start of treatment with inhaled IFN-β (SNG001 formulation) or placebo. Recovery is defined as ‘no limitation on activities’ or ‘no clinical or virological evidence of infection’. (Placebo n=6; SNG001 treatment n=6).

FIG. 6:

Changes in breathlessness status amongst those of the home cohort of SARS-CoV-2 infected patients exhibiting marked/severe breathlessness at the start of treatment (a) with provision of placebo (n=6) (b) with treatment with inhaled IFN-β (n=6).

FIG. 7:

Recovery for the combined hospital and home cohorts with or without marked/severe breathlessness at the start of treatment with inhaled IFN-β (SNG001 formulation) or placebo. Recovery is defined as ‘no limitation on activities’ or ‘no clinical or virological evidence of infection’ (a) Recovery for patients starting treatment with breathlessness score less than 3 (Placebo n=51; SNG001 n=47). (b) Recovery for patients starting treatment with a breathlessness score of at least 3 (Placebo n=36; SNG001 n=33; HR 3.41; 95% CI 1.47-7.94; p=0.004).

FIG. 8:

Recovery for the sub-groups of the hospitalised and home patient cohorts treated with SNG001 and placebo. Recovery is defined as ‘no limitation on activities’ or ‘no clinical or virological evidence of infection’. (a) Recovery for sub-group having breathlessness score of at least 3 or OSCI score of at least 3. (Placebo n=55; SNG001 n=54; HR 2.49; 95% CI 1.26-4.93; p=0.009) (b) Recovery for sub-group having breathlessness score of at least 3 or OSCI score of at least 4 (Placebo n=43; SNG001 n=48; HR 2.68; 95% CI 1.25-5.75; p=0.011).

FIG. 9:

Results showing the ability of IFN-β1a as present in the SNG100 formulation to inhibit the SAR2-CoV-2 designated “Wuhan-like” (Germany/BavPat1/2020) and the recognised variants of concern, Alpha variant (B.1.17) and Beta variant (B.1.351) at concentrations achievable by inhaled use.

### DETAILED DESCRIPTION

The use of inhaled IFN-β according to the present invention is currently primarily of proposed application in relation to preventing or reducing the severity of lower respiratory tract illness in a patient infected with the coronavirus SARS-CoV-2 and/or improving one or more symptoms and/or outcome in a patient so infected. However, SARS-CoV-2 is just one example of a coronavirus which has emerged in recent years as a causative agent of severe respiratory illness, commonly referred to as virus-induced acute respiratory distress syndrome (ARDS). Other prior known coronaviruses in this category include another severe acute respiratory syndrome coronavirus (SARS-CoV, also known as SARS-CoV-1) and Middle East Respiratory syndrome coronavirus (MERS virus). As indicated above, the invention is equally applicable to any such coronavirus, whether known or that might emerge in the future. Information well-known in the respiratory virus field on coronavirus-induced ARDS can be found by reference to, for example, Luyt et al. Virus-induced acute respiratory distress syndrome: epidemiology, management and outcome. Presse Med. 2011;40 (12 Pt 2):e561-8 and Horie et al. Emerging pharmacological therapies for ARDS: COVID-19 and beyond. Intensive Care Med. 2020; 46 (12)2265-2283.

The reason for the severity of respiratory disease caused by such coronaviruses is thought likely to be linked to their zoonotic origins (jumping from a non-human animal host to a human host, sometimes through an intermediate host) (Heeney et al. (2006) J. Intern. Med. 260, 399-408). In the absence of a vaccine, the human host lacks specific immunity towards the new pathogen providing the virus with an enhanced opportunity to infect and replicate within susceptible cells and to cause tissue injury. However, the overall risk is balanced by the ability of the virus to spread within the population. Unfortunately, in the case of SARS CoV-2, the virus is well adapted for human to human transmission and spreads quickly from person to person (Chan et al. A familial cluster of pneumonia associated with the 2019 novel coronavirus indicating person-to-person transmission: a study of a family cluster. Lancet. 2020;395 (10223):514-23). Consequently, by July 2020, the COVID-19 pandemic has resulted in around 14 million cases worldwide with approximately 600,000 deaths (https://covid19.who.int/) and, for many of the affected patients, there still remains a risk of long-term health consequences due to injury of their lungs and other organs (George et al. Pulmonary fibrosis and COVID-19: the potential role for antifibrotic therapy. Lancet Respir. Med. (2020) 8, 807-815). Furthermore, the lockdown measures taken to control spread of the virus around the world have had enormous economic consequences (https://www.ons.gov.uk/economy/grossdomesticproductgdp/articles/coronavirusandtheimpa ctonoutputintheukeconomy/may2020). Despite multiple targeted and non-targeted interventions reported, some of which have been discussed above, new treatments for COVID-19 remain a high priority requirement.

Although many people who are infected with SARS-CoV-2 are asymptomatic, spread of the virus into the lungs of susceptible individuals causes diffuse alveolar damage allowing leakage of fluid from capillaries into the alveolar space where it collects and limits normal exchange of oxygen and carbon dioxide resulting in respiratory failure (Buja et al. The emerging spectrum of cardiopulmonary pathology of the coronavirus disease 2019 (COVID-19): Report of 3 autopsies from Houston, Texas, and review of autopsy findings from other United States cities. Cardiovasc Pathol. 2020; 48:107233). On infection with SARS-CoV-2, the median incubation period is approximately 4-5 days before symptom onset with 97.5% of symptomatic patients developing symptoms within 11.5 days (see Guan et al. Clinical Characteristics of Coronavirus Disease 2019 in China. N. Engl. J. Med. (2020) 382 (18):1708-20; Lauer et al. The Incubation Period of Coronavirus Disease 2019 (COVID-19) From Publicly Reported Confirmed Cases: Estimation and Application. Ann. Intern. Med. 2020;172 (9):577-82; Pung et al. Investigation of three clusters of COVID-19 in Singapore: implications for surveillance and response measures. Lancet. 2020;395 (10229):1039-46 and Li et al. Early Transmission Dynamics in Wuhan, China, of Novel Coronavirus-Infected Pneumonia. N. Engl. J. Med. 2020;382 (13):1199-207).

COVID-19 patients usually present with fever, dry cough, shortness of breath, headache and malaise. Progression to pneumonia usually occurs 1-2 weeks after the beginning of the symptoms and involves decreased oxygen saturation, deterioration of blood gas, multi-focal glass ground opacities, or patchy/segmental consolidation in chest X-ray or CT. Severe COVID-19 cases progress to acute respiratory distress syndrome (ARDS), on average around 8-9 days after symptom onset (Huang et al. Clinical features of patients infected with 2019 novel coronavirus in Wuhan, China. Lancet. 2020;395 (10223):497-506; Wang et al. Clinical Characteristics of 138 Hospitalized Patients With 2019 Novel Coronavirus-Infected Pneumonia in Wuhan, China. JAMA. 2020, 323 (11): 1061-1069). SARS-CoV-2 infection in the lungs is accompanied by a profound downstream cytokine cascade known as a “cytokine storm” which suggests a hyperactive immune response whose dysregulation contributes to lung injury, ARDS, sepsis, organ failure and can be fatal in the most severe cases (Rageb et al. The COVID-19 Cytokine Storm; What We Know So Far. Front Immunol. 2020;11:1446). The invention is applicable to any coronavirus which presents a similar clinical picture associated with development of ARDS or alternatively referred to as severe acute respiratory syndrome.

Coronaviruses are a large family of enveloped RNA viruses that mostly infect birds and mammals. SARS-CoV-2 is a betacoronavirus with 79% genetic homology with SARS-CoV, and 98% homology to the bat coronavirus RaTG13 (Zhou et al. A pneumonia outbreak associated with a new coronavirus of probable bat origin, Nature, 2020;579 (7798):270-3). It is spread in respiratory droplets and infects nasal, bronchial and alveolar epithelial cells by binding of the viral spike protein to its cellular receptor, ACE2 (Walls et al. Structure, Function, and Antigenicity of the SARS-CoV-2 Spike Glycoprotein. Cell. (2020)180, 281-292).

Owing to the error prone nature of the viral replication process, RNA viruses such as SARS-CoV-2 accumulate mutations resulting in some sequence diversity. Nevertheless, different strains of SARS-CoV-2 can be recognised by sequencing and phylogenetic sequence trees. Exemplification of such phylogenetic tree analysis with rapid sequencing of isolates is reported for example by Meredith et al. Rapid Implementation of SARS-CoV-2 sequencing to investigate cases of health-care associated COVID-19: a prospective genomic surveillance study in The Lancet , published on—line 14 Jul. 2020. Sequences of amplified SARS virus genome can be compared with the NCBI Reference sequence NC_045512.2 or equivalent GenBank reference MN908947.3 for SARS-CoV-2 corresponding to the SARS-CoV-2 isolate Wuhan-Hu-1 complete genome (Wu et al. Nature 579, 265-269). SARS2-CoV-2 variant strains can thus be recognised and can be expected to have high homology to the reference genome, e.g. at least 90%, at least 95%, at least 98%, at least 99%. Such sequencing surveillance can equally enable any new SARS virus infecting humans to be identified. The use of inhaled IFN-β is seen as preferable in preventing or reducing the severity of lower respiratory tract illness in humans infected with any SARS-CoV virus, especially any SARS-CoV-2 viral strain.

### Nature of Interferon Beta for Administration

The term IFN-beta or IFN-β as used herein will be understood to refer to any form or analogue of IFN-β that retains the biological activity of native IFN-β and preferably retains the activity of IFN-β as present in the lung and in particular the pulmonary epithelium when induced by viral infection such as influenza or rhinovirus infection.

The IFN-β may be identical to or comprise the sequence of human IFN-β1a or human IFNβ-1b. However the IFN-β may also be a variant to such a native sequence, for example, a variant having at least 80%, at least 85%, at least 90%, at least 95-99% identity. It may have one or more chemical modifications provided the desired biological activity is retained.

The IFN-β will preferably be a recombinant IFN-β, e.g. produced in cells in vitro by expression of the polypeptide from a recombinant expression vector and purified from such culture.

Preferred is human recombinant IFN-β1a, e.g. as available from Rentschler Biopharma SE or Akron Biotechnology, LLC (Akron Biotech).

### Formulation and Mode of Administration

The IFN-β for administration by inhalation will generally be formulated as an aqueous solution, preferably at or about neutral pH, e.g. about pH 6-7, preferably, for example pH 6.5. Methods for formulating IFN-β for airway delivery in aqueous solution are well known, see for example U.S. Pat. No. 6,030,609 and European Patent no. 2544705. Preferably such an aqueous formulation will be employed which does not contain mannitol, human serum albumin (HSA) and arginine which are present in injectable IFN-β formulations. The composition may preferably contain an antioxidant such as methionine, e.g. DL-methionine. Such a ready-to-use formulation of IFN-β1a can also be obtained commercially, e.g. prepared in syringes at appropriate dilution of the IFN-β, e.g. from Vetter Pharma. It may conform with the formulation designated herein as SNG001 as previously used in clinical trials as referred to above in patients exhibiting viral exacerbation of asthma or COPD (subject to possible variation of the precise IFN-β1a concentration). Further details of this formulation are available in European Patent no. 2544705 and in the exemplification herein below. The concentration of IFN-β1a may be adjusted as discussed below. The precise preferred concentration of IFN-β, or more particularly IFN-β1a, may vary with the precise mode of delivery.

SNG001 has been shown to inhibit a broad range of viruses in cell-based assays. Of particular relevance, SNG001 has been shown to inhibit viral shedding following Middle East Respiratory Syndrome-coronavirus (MERS-CoV) infection in cell-based assays, with a similar potency to that reported in the literature and against other virus types. See also the cell-based assays reported in the exemplification which confirm the ability of SNG001 to have activity against various SARS-CoV-2 variants at inhaled delivery applicable concentrations. This reflects the general proposed mechanism underpinning the now proposed therapeutic use.

A pH neutral or about neutral pH IFN-β formulation e.g. pH 6.5, rather than a lower pH formulation, is especially favoured. A low pH is known to trigger cough. In Phase II trials of SNG001 in asthmatics, cough occurred in <10% of patients and the incidence was no different to that seen with placebo.

Delivery may be made using any device for aerosolization of a liquid formulation which retains the IFN-β activity, e.g. a nebuliser. Various nebulizers for drug delivery are commercially available and might be employed, e.g. the I-neb or Ultra nebuliser made by Philips Respironics and Aerogen respectively. Both devices have been shown to enable convenient inhalation delivery of IFN-β1a with retention of IFN-β activity after aerosolization.

### Dosage

A suitable IFN-β dose for any inhalation delivery mode may be established by a dose escalation study with assessment of induced anti-viral response in the lungs, generally a dose which ensures a robust anti-viral response within 24 hours after dose administration, preferably so as to support a once-a-day dosing regimen. This may be assessed by reference to appropriate biomarkers.

For nebuliser delivery of an aqueous formulation containing IFN-β1a, an aqueous formulation as discussed above containing about 11-13 MIU/ml IFN-β1a, e.g. 11-12 MIU/ml may be found suitable.

A suitable once-a-day dosing schedule has been achieved by delivering 0.5 ml or about 0.5 ml of an aqueous formulation containing IFN-β1a at about 11-12 MIU/ml, preferably 12 MIU/ml IFN-β1a, from the I-neb nebuliser (Phillips Respronics) and may be found suitable with other nebulisers providing similar efficiency of airway delivery. If using alternative nebulisers, the dose may need to be adjusted to take into account differences in efficiency of drug delivery to the lungs. Once daily delivery may preferably be carried out. Delivery may be over a number of days, e.g. for 3 or more days, for 5 or more days or 7 or more days, e.g. up to 14-15 days or longer to alleviate LRT illness and preferably step improvement in score back to a lower score, e.g. recovery down to at least OSCI score 1.

### Timing of Administration

As noted above, the previous indication has been that, if considered at all for reducing LRT illness in patients associated with coronavirus infection capable of causing acute respiratory distress syndrome (ARDS), e.g. severe acute respiratory syndrome as observed in COVID-19 patients following infection with SARS-CoV-2, administration of IFN-β is recommended as confined to early intervention from the on-set of symptoms, more particularly ahead of 7 days and that dosage should if anything be graduated down ahead of that time point (see again Hung et al. (2020) Lancet 395: 1695-16704 entitled ‘Triple combination of interferon beta-1b, lopinavir-ritonavir and ribavirin in the treatment of patients admitted to hospital with covid-19: an open-label, randomised, phase 2 trial’; first published on-line 8 May 2020). In contrast, the data now presented not only establishes for the first time that administration of IFN-β by inhalation can be beneficial in alleviating LRT illness arising from SAR2-CoV-2 infection but that such intervention can be beneficial even with patients presenting with symptoms equating with LRT illness such that a clinical score of at least 3 or even at least 4 must be assigned on the Ordinal Scale for Clinical Improvement. Such patients will commonly have established LRT illness warranting hospitalisation and will commonly have experienced symptoms of SARS-CoV-2 infection affecting activity. They will commonly have had symptoms of such infection for at least 7 days. For those patients in the study herein reported, the median time from start of symptoms to start of treatment with inhaled IFN-β was over 9 days.

Thus in accordance with the invention, IFN-β, e.g. recombinant IFN-β1a, may be administered by inhalation to a patient with a virus infection, more particularly for example a coronavirus infection, capable of causing severe LRT illness, e.g. SARS-CoV-2, at a stage corresponding with a score of at least 3 or at least 4 on the WHO Ordinal Scale for Clinical Improvement. Use may encompass patients 7 days or more post onset of symptoms of viral infection, e.g. 9 or more days post onset of symptoms of viral infection. Generally, such administration will be before the patient reaches score 5 or before the patient reaches score 6. Preferably administration will be before any mechanical ventilation. Such administration has been shown compared with placebo to provide greater probability of prevention of the patient progressing to a higher score, e.g. need for intensive care and/or non-invasive ventilation or possibly mechanical ventilation, and to rather encourage back-stepping to a lower score. Desirably administration of inhaled IFN-β will be accompanied by no increase of score.

Effectiveness may be assessed daily by assessing breathlessness or obtaining a BCSS score. Generally administration will be continued with improvement in breathlessness or BCSS score. Desirably, it will be with attainment of one or more steps down on the WHO Ordinal Scale for Clinical Improvement, preferably a reduction of score of one step or more within, for example, 14 days or less, less than 7 days, more preferably less than 6 days, for example 5 days, still more preferably less than 4 days, e.g. 3 days. Desirably, administration of inhaled IFN-β will be accompanied by attainment of no limitation on activities or no clinical or virological evidence of infection, e.g. within 14 days or less of IFN-β administration. The overall desired outcome will be earlier release from hospital than anticipated with no treatment beyond supplemental oxygen supply by mask or nasal prongs.

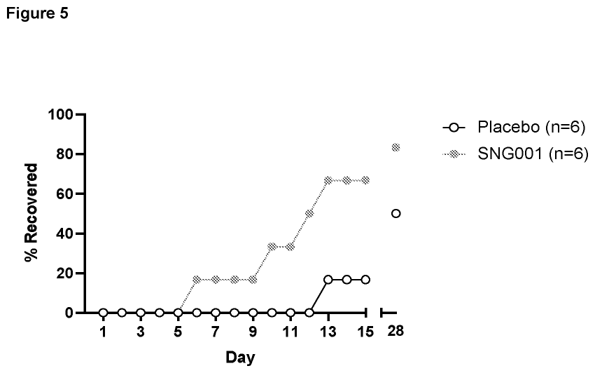

As indicated above, it has been found that preferable targeting of inhaled IFN-β treatment may be on the basis of breathlessness score, more particularly a breathlessness score of 3 or 4 (equating with marked or severe breathlessness). This translates to the home environment and SARS-Cov-2 infected individuals having a OSCI score where the desire is rapid improvement from an OSCI score of 2 (equating with limitation of activities) down to an OSCI score of 1 or 0 without need for hospitalisation. See FIG. 5 and the exemplification.

Thus now proposed is inhaled IFN-β for use in treating patients in accordance with the invention who are suffering illness as a result of viral infection, more particularly for example SARS-CoV-2 infection, wherein a breathlessness score of 3-4 is used as the determiner for administration of inhaled IFN-β in either the hospital or home environment.

Such simple point of care targeting of inhaled IFN-β treatment to SARS-CoV2 infected patients based on single symptom scoring (breathlessness) is thus now indicated as an important contribution to clinical management of such patients which was not predictable from any previous knowledge of interferon Type I action.

### Combination Therapy

Although the data now presented supports use of inhaled IFN-β as a sole therapeutic agent to prevent or reduce severity of LRT illness in a coronavirus infected patient as discussed above, it will be appreciated that such administration of IFN-β is not excluded with one or more other therapeutic agents which may assist improvement of one or more symptoms of the patient arising from the viral infection. Use of inhaled IFN-β may be combined for example with administration of a corticosteroid, e.g. dexamethasone, or any other agent previously proposed for preventing or reducing the severity of LRT illness in coronavirus-infected individuals liable to present with ARDS e.g. lopinavir-ritonavir and/or ribavirin or intravenous remdesivir (an RNA polymerase inhibitor that has been indicated to shorten time to hospital discharge of COVID-19 patients but does not reduce respiratory tract viral load). Such combined therapy may involve simultaneous, sequential or separate administration of IFN-β and another therapeutic agent as appropriate. Of especial interest may be a combination of inhaled IFN-β with dexamethasone (The RECOVERY Collaborative Group, Dexamethosone in Hospitalised Patients with Covid-19 -Preliminary Report, N. England J. Med. 17 Jul. 2020). Of especial interest may be administration additionally of an inhaled corticosteroid. Combined use of such a corticosteroid and IFN-β, e.g. as a single pharmaceutical composition for aerosol delivery to the airways, has previously been proposed for use in alleviating viral exacerbation of asthma or COPD (See EP1734987B).

### Method of Treatment

In another aspect, the invention provides a method of preventing or reducing the severity of lower respiratory tract illness in a patient infected with a coronavirus or other potentially pandemic-causing virus capable of causing acute respiratory distress syndrome (ARDS) and/or improving one or more symptoms and/or outcome in a patient so infected, wherein said method comprises administering IFN-β by inhalation. The IFN-β may be administered as a sole therapeutic agent or in combination with one or more further therapeutic agents to assist improvement of one or more symptoms arising from the same viral infection as discussed above. As indicated above such administration may be preferably preceded by determination of a breathlessness score equating with marked or severe and may be in the home or hospital environment with a view to accelerating recovery, preferably down to at least 1 on the OSCI.

The invention also provides use of IFN-β for the manufacture of a composition for use in a method of preventing or reducing the severity of lower respiratory tract illness as disclosed herein wherein the composition is administered by inhalation. As indicated above, such administration will generally employ a device for aerosolization of a liquid formulation which retains the IFN-β activity, e.g. a nebuliser.

### EXAMPLE

Comparison of the efficacy and safety of inhaled interferon-beta to placebo administered to patients hospitalised with COVID-19 caused by SARS-CoV-2.

### Summary of Protocol

This was a randomised, double-blind, parallel, placebo-controlled trial of inhaled recombinant IFN-β1a formulated as an aqueous formulation at neutral pH for delivery by nebulisation to patients with confirmed SARS-CoV-2 infection. 98 hospitalised patients with confirmed SARS-CoV-2 infection (recruited from 9 specialist hospital sites in the UK during the period 30 March to 27 May 2020) were randomised for 14-day treatment with inhaled IFN-β (n=48) or placebo (n=50). Patients were randomised within 24 hours of a positive test result for first treatment (unless the positive test result occurred prior to hospitalisation). The inclusion criteria included patients being admitted to hospital due to the severity of the COVID-19 disease, being aged ≥18 years and having SARS-CoV-2 infection (determined by either a positive RT-PCR test result or a positive point of care test in the presence of strong clinical suspicion).

Patient groups were evenly matched in terms of average age (56.5 years for placebo and 57.8 years for SNG001), comorbidities and average duration of COVID-19 symptoms prior to enrolment (9.8 days for placebo and 9.6 days for SNG001).

Patients received inhaled IFN-β or placebo (formulation buffer without IFN-β) from a portable mesh nebuliser (I-neb as supplied by Philips Respironics, Chichester, UK). The primary end point was prevention of severe lower respiratory tract illness as determined by step movement within the 9-point Ordinal Scale for Clinical Improvement.

### Formulation of Recombinant IFN-β1a

The formulation (referred to as SNG001) provides recombinant IFN-β1a (manufactured by Rentschler Biopharma SE or Akron Biotechnology, LLC) formulated as an aqueous solution buffered at pH 6.5. The composition is set out in the table below. Unlike some other commercial preparations, it does not contain mannitol, human serum albumin or arginine. The formulation was provided in ready-to-use syringes by Vetter Pharma.

SNG001 formulation:

| Ingredient | Quantity (per ml) | | Function |
|---|---|---|---|
| IFN-β1a | About 12 MIU/ml | | Active ingredient |
| Sodium dihydrogen | 5.92 | mg | Buffer component |
| phosphate dihydrate | | | |
| Disodium phosphate | 2.13 | mg | Buffer component |
| dihydrate | | | |
| Sodium citrate | 20.58 | mg | Chelating agent, buffer |
| | | | component |
| Methionine | 0.30 | mg | Stabiliser, antioxidant |
| Water | 1 | ml | solvent |

As noted above, SNG001 has been shown to inhibit a broad range of viruses in cell-based assays. Of particular relevance, SNG001 has been shown to inhibit viral shedding following Middle East Respiratory Syndrome-coronavirus (MERS-CoV) infection in cell-based assays, with a similar potency to that reported in the literature and against other virus types (Scagnolari et al. Increased sensitivity of SARS-coronavirus to a combination of human type I and type II interferons. Antivir. Ther. 2004 December;9 (6):1003-11; Sheahan et al. Comparative therapeutic efficacy of remdesivir and combination lopinavir, ritonavir, and interferon beta against MERS-CoV. Nat. Commun. 2020 Jan. 10;11 (1):222; Spiegel et al. The antiviral effect of interferon-beta against SARS-coronavirus is not mediated by MxA protein. J. Clin. Virol. 2004 July;30 (3):211-3).

In all previous clinical trials of inhaled SNG001 (3 in asthma and one in chronic obstructive pulmonary disease [COPD]), it was shown to upregulate lung antiviral biomarkers in sputum for 24 hours after dosing, confirming successful delivery of biologically active drug to the lungs, demonstrating proof-of-mechanism and supporting dose selection.

A dose escalating trial with the chosen nebuliser established a target lung dose which induced an antiviral response in the lungs that was present 24 hours after dose administration.

### Administration Protocol

The SNG001 nebulizer solution was presented in glass syringes containing 0.65 ml of IFN-β1a aqueous solution with an IFN-β1a concentration of 12 MIU/ml. The I-neb nebulizer, fitted with a 0.53 ml chamber, was filled with the contents of 1 syringe. Patients inhaled one dose per day or placebo solution. The nebulizer was used in its tidal breathing mode. In this mode, the I-neb delivers short pulses of aerosol into each inhalation and requires the patient to use tidal breathing.

### Respiratory and Other Assessments

In addition to assessment once a day with reference to the Ordinal Scale for Clinical Improvement, patients in the trial were also subject to BOSS assessment and assessment for pneumonia.

### BCSS Assessment

The BCSS is a patient-reported outcome measure that was designed as a daily diary in which patients are asked to record the severity of three symptoms: breathlessness, cough and sputum.

Each symptom is represented by a single item which is evaluated on a 5-point scale ranging from 0-4, with higher scores indicating more severe symptoms. Total score is expressed as the sum of the three-item score, with a range of 0-12. A mean decline of 1 point on the BCSS total scale signifies a substantial reduction in symptom severity.

This assessment was carried out once a day at the same time each day (+/−3 hours). It was completed by the patient where possible. However, if needed, site staff read out the questions to the patient either face-to-face or over telephone/video link.

The BCSS questions and possible responses are as follows:
1. How much difficulty did you have breathing today?
0=None—unaware of any difficulty
1=Mild—noticeable when performing strenuous activity (e.g. running)
2=Moderate—noticeable even when performing light activity (e.g. bedmaking or carrying groceries)
3=Marked—noticeable when washing or dressing
4=Severe—almost constant, present even when resting
2. How was your cough today?
0=No cough—unaware of coughing
1=Rare—cough now and then
2=Occasional—less than hourly
3=Frequent—one or more times an hour
4=Almost constant—never free of cough or need to cough
3. How much trouble did you have due to sputum today?
0=None—unaware of any trouble
1=Mild—rarely caused trouble
2=Moderate—noticeable trouble
3=Marked—caused a great deal of trouble
4=Severe—almost constant trouble

### Findings Supporting Benefit of IFN-β Administration to COVID-19 Patients

The odds of patients developing severe disease during the treatment period were reduced by 72% in patients treated with SNG001 compared to the placebo group. More particularly, the odds of patients developing severe disease (e.g. requiring ventilation or resulting in death) during the treatment period were markedly reduced by 72% for patients receiving SNG001 compared to patients who received placebo (OR 0.28 [95% CI 0.07-1.08]; p=0.064)

The trial results showed that SNG001 greatly reduced the number of hospitalised COVID-19 patients who progressed from requiring oxygen (Score 4 on the WHO Ordinal Scale for Clinical Improvement) to requiring ventilation (Score 5 or above).

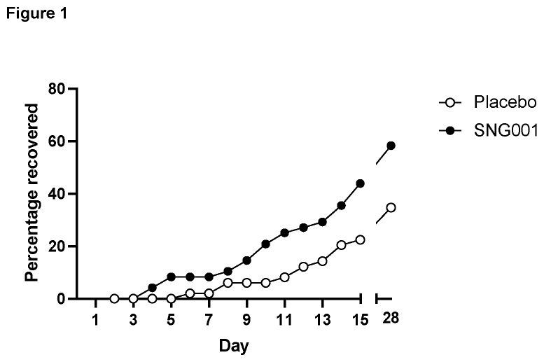

Patients who received SNG001 were more than twice as likely to recover (defined as ‘no limitation on activities’ or ‘no clinical or virological evidence of infection’) from COVID-19 as those receiving placebo (HR 2.19 [95% CI 1.03-4.69]; p=0.043). See FIG. 1.

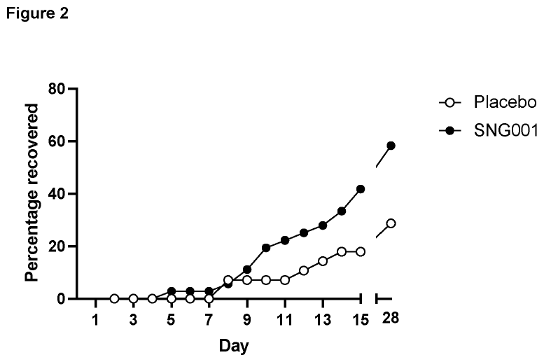

In the case of patients with more severe disease at time of admission (i.e. requiring treatment with supplemental oxygen), patients treated with SNG001 (n=28) were more than twice as likely to have recovered by the end of the treatment period and had greater odds of recovery at day 28 than the placebo group (n=36). See FIG. 2. Again recovery was defined as ‘no limitation on activities’ or ‘no clinical or virological evidence of infection’

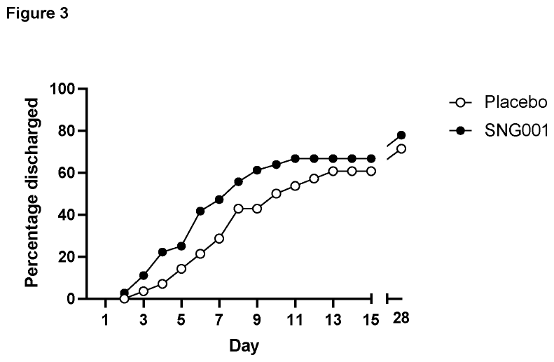

In the patients with more severe disease at time of admission (i.e. requiring treatment with supplemental oxygen), treatment with SNG001 increased the likelihood of hospital discharge. The median time to discharge was 6 days for patients treated with SNG001 and 9 days for those treated receiving placebo. See FIG. 3

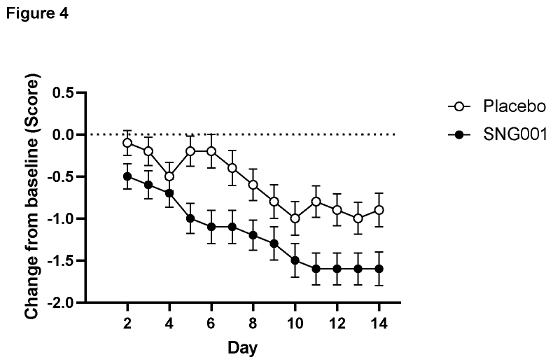

Over the treatment period, breathlessness, one of the main symptoms of severe COVID-19, was markedly reduced in patients who received SNG001 compared to those receiving placebo (p=0.007). See FIG. 4.

Three subjects died after being randomised to placebo. There were no deaths among subjects treated with SNG001.

As noted above, by day 28, patients with more severe disease at the time of admission (i.e. requiring treatment with supplemental oxygen) who received SNG001 treatment had significantly better odds of recovery (OR 3.86 [95% CI 1.27-11.75]; p=0.017).

Interestingly, the efficacy analyses indicate that there is no evidence of an association between the inhaled SNG001 treatment effect and prior duration of COVID-19 symptoms. This is contrary to previous suggestion that use of IFN-β should be confined to early intervention from on-set of symptoms, less than 7 days.

### Administration of Inhaled IFN-β to SARS-CoV-2 Infected Individuals in the Home Environment

The above discussed trial in hospitalised patients was extended to a cohort of 120 SARS-CoV-2 infected patients in the home environment. All patients were deemed at risk patients for progression to need for hospitalization (>65 years of age, or >50 years of age with risk factor). All participants in the trial were either at score 1 (no limitation of activities) or score 2 (limitation of activities but not requiring hospitalization) on the WHO Ordinal Scale at the start of treatment. SNG001 or placebo was inhaled again by mesh nebuliser once a day. Remote device training was provided to participants with the trial being conducted using video calls.

Participants were assessed for deterioration and, where initially at score 2, recovery to score 1 or 0 without rebound. Only two patients deteriorated to score 3 or above and both were on placebo. This was in accordance with expectation for at risk SARS-CoV-2 infected individuals.

In addition to overall disease score on the WHO ordinal scale being assessed daily, participants in the trial were also scored daily for breathlessness on the breathlessness scoring scale as set out above and reiterated below:

### Breathlessness Scoring in Response to the Question: How Much Difficulty Did You Have Breathing Today?

0=None—unaware of any difficulty
1=Mild—noticeable when performing strenuous activity (e.g. running)
2=Moderate—noticeable even when performing light activity (e.g. bedmaking or carrying groceries)
3=Marked—noticeable when washing or dressing
4=Severe—almost constant, present even when resting.

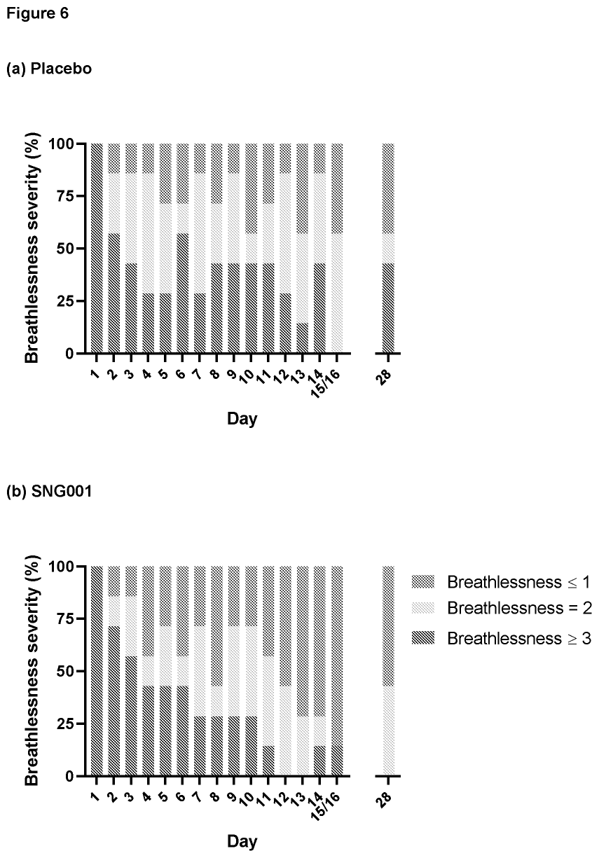

Of particular interest was the unpredictable finding that, for the 11% of patients with a breathlessness score of at least 3 at the start of treatment, the effect of SNG001 compared to placebo on promoting recovery was clear as shown in FIG. 5. The ability of SNG001 compared to placebo to significantly promote improvement of breathlessness status in patients with marked or severe breathless at the start of treatment is also illustrated by FIGS. 6a and 6b.

Hence, inhaled IFN-β is pointed to as a useful point of care treatment for SARS-CoV-2 patients experiencing marked to severe breathlessness at home with a view to accelerating recovery and reducing risk of progression to need for hospitalization.

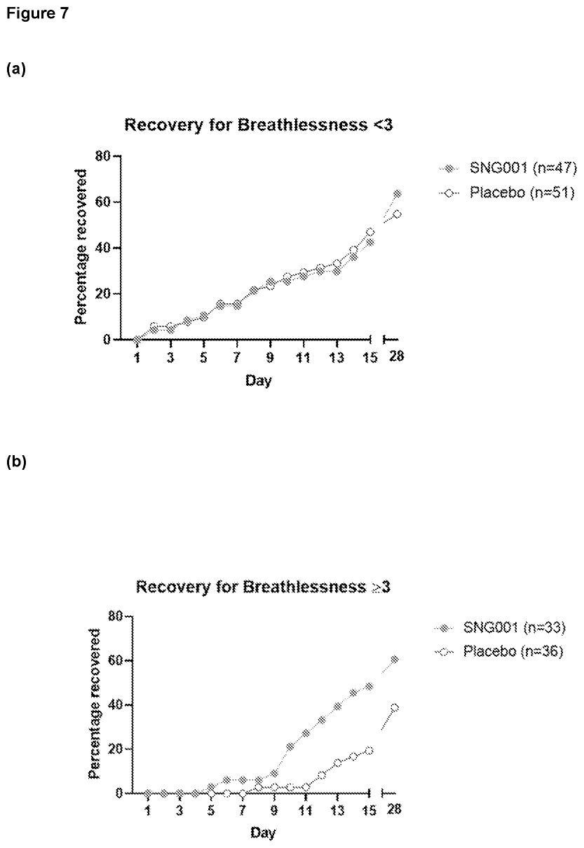

Moreover, by combining data from hospital and home study cohorts, it is now indicated that the same breathlessness scoring (a score of 3-4 equating with marked or severe breathlessness) is a simple and quick criterion which may be usefully employed in targeting COVID-19 patients whether in the hospital environment or at home, for treatment with inhaled IFN-β with a view to accelerating recovery. Whilst no significant difference was observed in recovery rate between SNG001 and placebo treatment in patients with a breathlessness score of 0, 1 or 2, patients scoring 3 or 4 for breathlessness receiving SNG001 were found to exhibit significantly accelerated recovery compared to such patients receiving placebo. See FIGS. 7a and 7b.

Patients scoring 3 or 4 for breathlessness and administered SNG001 were found to be more than 3 times as likely to recover than patients taking placebo [HR 3.41 (95% CI; 1.47, 7.94) p=0.004].

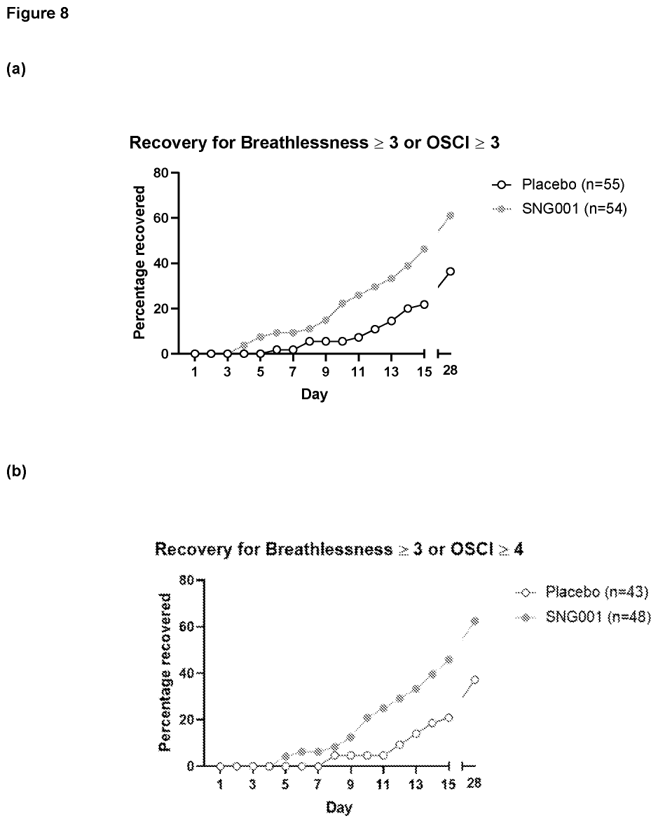

The utility of simple breathlessness scoring for categorising COVID-19 patients for treatment with inhaled IFN-β to promote recovery is further supported by FIGS. 8a and 8b which show percentage recovery with treatment (SNG001 or placebo) for sub-groups of hospitalised and home patients based on overall disease severity score on the WHO Ordinal Scoring Scale or a breathlessness score of at least 3: Group 1: breathlessness score at least 3, or Ordinal Score at least 3; Group 2: breathlessness score at least 3, or Ordinal score at least 4. Those exhibiting marked breathlessness or an OSCI score of 3 or 4 showed a highly significant effect of SNG001.

Targeting inhaled IFN-β treatment to SARS-CoV2 infected patients based on simple breathlessness scoring is thus now indicated as an important contribution to clinical management of such patients which was not foreshadowed by previous knowledge on any action of such interferon. Targeting of inhaled IFN-β treatment in hospitalised COVID-19 patients on the basis of markedly/severe breathlessness is indicated as a means for promoting recovery which can be translated to COVID-19 patients at home with breathlessness problems but not yet exhibiting overall disease symptom severity warranting need for a hospital bed.

### In Vitro Studies Confirming Ability of IFN-β1a as Present in the Inhaled Formulation to Inhibit SARS-CoV-2 Variants

To supplement the above discussed trials, in vitro experiments were conducted to confirm that SNG001 at concentration achievable by inhaled delivery has activity against both the

Alpha and Beta variants of SAR2-CoV-2 (as now designated by the WHO Greek alphabet labelling scheme for such variants), as well as “Wuhan-like” SAR2-CoV-2 (Germany/BavPat1/2020). The Alpha and Beta variants were previously referred to as B. 1.1.7 and B1.351 respectively or commonly respectively as the UK Kent variant and the South African variant. See the WHO website and notice issued 31 May 2021 re designation of SAR2-CoV-2 variants.

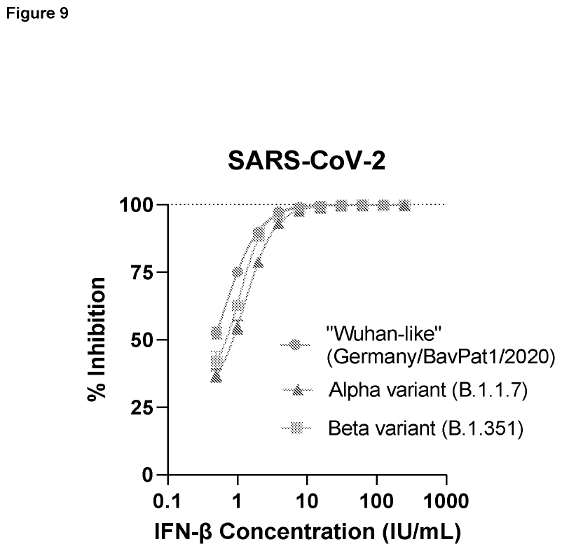

Vero E6 cells were treated with the SNG001 formulation at various concentrations prior to and after infection with SARS-CoV-2 as noted above. 16-24 hrs after infection the presence of SAR2-CoV-2 viral proteins was determined using an immunostaining method. SNG001 potently reduced virus to undetectable levels in cells infected with “Wuhan-like” virus or either of the above-noted variants. Concentrations readily achievable following inhaled delivery of IFN-β that were found to give 90% inhibition (IC₉₀) were 3.2, 3.4 and 4.0 IU/ml as shown in FIG. 9.

This data further supports use of inhaled IFN-β as claimed in preventing or reducing the severity of lower respiratory tract illness associated with SARS-CoV-2 virus, extending to known variants and possible future emerging variants, through general mechanism as an inhaled broad-spectrum anti-viral product.

1. A method for preventing or reducing the severity of lower respiratory tract illness in a patient infected with a coronavirus or other potentially pandemic-causing virus capable of causing acute respiratory distress syndrome (ARDS) and/or improving one or more symptoms and/or outcome in a patient so infected, which comprises administering interferon-beta (IFN-β) by inhalation.

2. The method according to claim 1 wherein said patient is infected with a coronavirus.

3. The method according to claim 2 wherein said coronavirus is a SARS virus capable of causing severe acute respiratory syndrome.

4. The method according to claim 3 wherein said coronavirus is SARS-CoV-2

5. The method according to claim 1 wherein the IFN-β is recombinant human IFN-β1a.

6. The method according to claim 1 wherein the IFN-β is formulated in an aqueous solution at about pH 6-7, e.g. pH 6.5, and preferably omitting mannitol, human serum albumin and arginine.

7. The method according to claim 1 wherein administration of IFN-β to the airways comprises aerosolization of a liquid formulation of the IFN-β.

8. The method according to claim 7 wherein said administration is by use of a nebuliser.

9. The method according to claim 1 wherein administration of the IFN-β is as a single inhaled dose daily.

10. The method according to claim 1 wherein the patient is assessed as having a score of at least 3 or at least 4 on the World Health Organization (WHO) Ordinal Scale for Clinical Improvement (OSCI) upon first administration of IFN-β.

11. The method according to claim 10 wherein the patient does not increase score on the same scale following IFN-β administration.

12. The method according to claim 11 wherein the patient exhibits a reduction of score of one or more on the same scale within the time period of IFN-β administration, preferably a reduction of score of one or more within 14 days or less, for example, less than 7 days, more preferably less than 6 days, for example 5 days, still more preferably less than 4 days, e.g. 3 days

13. The method according to claim 1 wherein improvement in breathlessness and/or Breathlessness, Cough and Sputum Score (BCSS) is observed following the start of IFN-β administration.

14. The method according to claim 1 wherein the patient is assessed as having a breathlessness score of 3 to 4 upon first treatment with IFN-β, said score being determined on the basis of response of the patient to query re degree of difficulty of breathing on the following scale:
0=None—unaware of any difficulty
1=Mild—noticeable when performing strenuous activity (e.g. running)
2=Moderate—noticeable even when performing light activity (e.g. bedmaking or carrying groceries)
3=Marked—noticeable when washing or dressing
4=Severe—almost constant, present even when resting.

15. The method according to claim 14 wherein a breathlessness score of 3-4 is used as the determiner for administration of inhaled IFN-β.

16. The method according to claim 15 wherein the patient is in the home environment equating with an OSCI score of no more than 2 and breathlessness score of at least 3 is used as the determiner for administration of inhaled IFN-β.

17. The method according to claim 1 wherein the IFN-β is administered by inhalation in combination with administration of one or more further therapeutic agents to assist improvement of one or more symptoms arising from the same viral infection, wherein each further agent is administered simultaneously, separately or sequentially.

18. The method according to claim 17 wherein IFN-β is administered in combination with a corticosteroid.
